(19) United States
(12) Patent Application Publication
(10) Pub. No.: US 2007/0007468 A1
(43) Pub. Date: Jan. 11, 2007

(54) Inhomogeneous magnetic field cyclotron

(76) Inventor: Willard H. Schmidt, Albuquerque, NM (US)

Correspondence Address: Willard H. Schmidt, 2015 Altez NE, Albuquerque, NM, 87112, US

(21) Appl. No.: 11/175,722

(22) Filed: Jul. 7, 2005

(51) Int. Cl. A61N 5/00

(52) U.S. Cl. 250/492.3

(57) ABSTRACT

This patent is for an inhomogeneous magnet field cyclotron that has no moving parts and no operating oscillating magnetic or electrical fields. This device or machine consists of any number of individual magnets designed to produce a strong inhomogeneous magnetic field with magnets placed front to back in a closed geometrical arrangement or ring called a “racetrack”. Electrical neutral particles with a magnetic moment are injected into the magnets and pass through a tunnel in each of the magnets and around the racetrack. The subject particles can be either accelerated or decelerated in the racetrack depending on the direction of travel, orientation of magnets, and the sign of particle magnetic moment. As a result of the inhomogeneous magnetic field gradient, a particle in the field experiences a magnetic force due to its magnetic moment. The net direction of the magnetic force on particles is along a line between the opposing magnetic poles. The force on a particle is manifest each time the particle passes through the inhomogeneous magnetic field of a pair of magnets. Multiple passes around the racetrack result in significant acceleration or deceleration of subject particles. Without use of refrigerants, the kinetic temperature equivalent for particle velocities in the racetrack can be reduced to approximate absolute zero. Consequently, this cyclotron is a unique and versatile piece of research and industrial equipment.

### BACKGROUND—DESCRIPTION OF PRIOR ART

Between 1921 and 1924 Stem and Gerlach conducted laboratory experiments in Germany to study the effects of an inhomogeneous magnetic field on silver atoms. They discovered that the inhomogeneous magnetic field gradient induces a force on the atom due to the magnetic moment of the atom. Their experimental results verified the concept and numerical value of the Bohr magneton, and also proved certain quantum mechanics concepts.

Other scientists have used the inhomogeneous magnetic field concept and laboratory test arrangement to determine the magnetic moments of many kinds of atoms such as sodium and potassium, in addition to silver.

### DRAWING FIGURES

FIG. 1 is a top cross section view of the racetrack for the Inhomogeneous Magnetic Field Cyclotron

FIG. 2 is a top cross section view of several of the magnets that comprise the racetrack.

FIG. 3 is an isometric view of one of the magnets in the racetrack.

### REFERENCE NUMERALS IN DRAWINGS

1. Magnetic purveyor, or magnet

2. Particle pathway through magnet

3. Particle port (inlet)

4. Particle port (outlet)

5. Simulated invisible magnetic field lines

6. Particle containment tube (non-magnetic) through and between magnets

7. Particle port in rear face magnet

8. Particle port cut out

### DESCRIPTION OF INVENTION

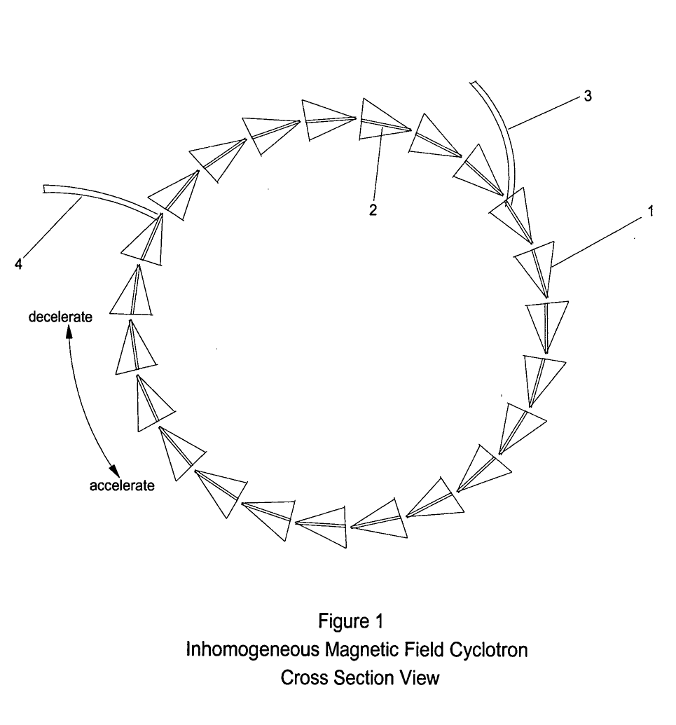

FIG. 1 is a cross section top view of the Inhomogeneous Magnetic Field Cyclotron. In total it is called the “racetrack”. The front to back combination of individual magnets 1 makes possible a highly inhomogeneous magnet field because the sharp edge of one magnet faces the blunt face of the one in front of it. The sharp edge of the magnet 1 is the north magnetic pole, and the blunt face is the south magnetic pole, or the magnetic polarity may be reversed. The strength of the magnets is due to residual magnetism, or the magnetic field can be enhanced by electric magnetic induction. The magnets may be made from any material that can support a magnetic field.

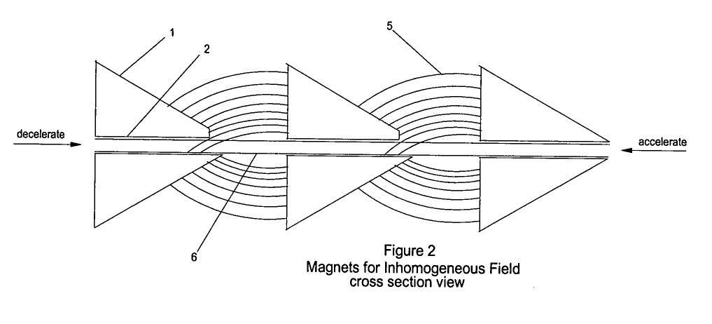

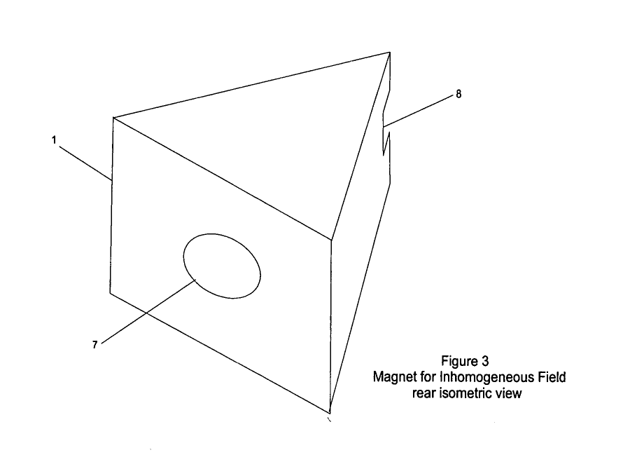

Each magnet 1 has a passageway through it from front to back 2 that permits particles to go from one magnet to the next and thereby travel completely around the racetrack. The particles are confined within a high vacuum non-magnetic particle containment tube 6 that goes through each of the magnets 1 and around the racetrack. Although particle ports 3,4 are shown on the drawing to indicate means for inlet and outlet of particles from the racetrack, each operational setup will have its own unique design and capabilities. In FIG. 2 the invisible magnetic field lines 5 are simulated in the drawing. In order to assure a strong magnetic field gradient across the particle path, a small section of one side of the sides of the magnet 1 knife edge is cut back to permit the magnetic field lines 5 to go across particle containment tube 6, as indicated in FIG. 2. The isometric drawing in FIG. 3 illustrates the particle port 7 in the back face of a magnet 1. The resultant particle port cutout 8 is evident on the knife-edge at the front of the magnet 1.

Operation of Inhomogeneous Magnetic Field Cyclotron

This subject Cyclotron operates by having electrically neutral particles introduced or fed into the racetrack so that the initial velocity carries them either counter-clock wise or clockwise around the racetrack depending on whether the particle is to be decelerated or accelerated. This cyclotron is a passive device that has no moving parts and no operating oscillating electrical or magnetic fields. The path of particles in this device is through the interior of the magnets perpendicular to the magnet knife edge, and not primarily along the knife-edge as in the Stem-Gerlach experiments and other magnetic moment experiments. The injected particles, with a magnetic moment, are subject to a magnetic force each time they encounter the inhomogeneous magnetic field between magnet pairs in the racetrack. The force on the particles is very small and is due to an interaction between the particle magnetic moment and the gradient of the inhomogeneous magnetic field between magnet pairs in the racetrack. The particles may cycle around the racetrack as many times as necessary to achieve specified criteria. In the deceleration mode, particles may reach velocities that approach the equivalent kinetic temperature of absolute zero without use of a refrigerant of any kind. Consequently, the probability of particle interactions becomes very high, and this cyclotron can serve as a valuable research and industrial piece of equipment.

### SUMMARY

The subject Inhomogeneous Magnetic Field Cyclotron can focus and decelerate or accelerate electrically neutral particles because of the interaction between the magnetic moment of the particles and the inhomogeneous magnetic field. The Cyclotron consists of front to back pairs of magnets in a closed geometrical path called a racetrack. The Cyclotron has no moving parts and no operating oscillating magnetic or electrical fields. After many cycles around the racetrack, particles may be decelerated to velocities that approach the kinetic equivalent of absolute zero. This Cyclotron can be a unique research and industrial piece of equipment.

I claim:

1. An inhomogeneous magnetic field cyclotron.

2. The cyclotron of claim 1 comprising a closed geometrical ring, or any closed geometrical shape or configuration, of magnet pairs called a “racetrack”.

3. The cyclotron of claim 1 wherein a pair of magnets adjacent to each other are placed front to back to create an inhomogeneous magnetic field between them.

4. The cyclotron of claim 1 comprising a particle containment tube that runs through each of the magnets and around the racetrack.

5. A racetrack with pairs of magnets that create an inhomogeneous magnetic field between them with which the magnetic moment of particles interact and generate a force perpendicular to the knife edge of one magnet and parallel to the magnetic poles between magnets to move the particles along the racetrack.

6. The racetrack of claim 5 comprising an inlet portal and an outlet portal for racetrack particles.

7. The racetrack of claim 5 comprising a series of magnet pairs around which particles with a magnetic moment and going in one direction are decelerated and particles going in the opposite direction are accelerated.

8. The racetrack of claim 5 comprising a series of inhomogeneous magnet fields that can decelerate particles with a magnetic moment to velocities that approach the equivalent kinetic temperature of absolute zero without use of a refrigerant.

9. The racetrack of claim 5 comprising multiple inhomogeneous magnetic fields in series that can accelerate or decelerate electrically neutral particles, depending on the sign of the magnetic moment and particle velocity direction around the racetrack, in order to focus and separate particles according to the sign and magnitude of the magnetic moments.

10. A magnet that has a long knife edge at one end for a magnetic pole and a flat face at the opposite magnetic pole on the other end.

11. A magnet of claim 10 wherein there exists an opening or tunnel through said magnet from the knife edge at one magnetic pole to the flat face at the opposite magnetic pole.

12. A magnet of claim 10 comprising a magnetic pole located at each end whose polarity can be either fixed or reversed as desired.

13. A magnet of claim 10 wherein one of the sides of the tunnel running through the magnet is cut back along the knife-edge of the magnet to enhance the magnetic field gradient at that location.
